(19) United States
(12) Patent Application Publication
(10) Pub. No.: US 2009/0299265 A1
(43) Pub. Date: Dec. 3, 2009

(54) Electrode Assembly for Iontophoresis Having Shape-Memory Separator and Iontophoresis Device Using the Same

(75) Inventors: Akihiko Tanioka, Ohota-ku (JP); Mizuo Nakayama, Shibuya-ku (JP); Takehiko Matsumura, Shibuya-ku (JP); Hidero Akiyama, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: TTI ellebeau, Inc., Tokyo (JP)

(21) Appl. No.: 11/992,672

(22) PCT Filed: Oct. 2, 2006
(86) PCT No.: PCT/JP2006/319684

§ 371 Date: Aug. 12, 2009

(30) Foreign Application Priority Data
Sep. 30, 2005 (JP) 2005-288618

(51) Int. Cl. A61N 1/30

(52) U.S. Cl. 604/20

(57) ABSTRACT

An electrode assembly for iontophoresis including an electrode coupled to an electric power source device having a same polarity as that of the ionic drug in the electrode assembly, an electrolyte solution holding portion holding an electrolyte solution by being impregnated with the electrolyte solution, the electrolyte solution holding portion being placed adjacent to the electrode, a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion, a drug solution holding portion holding the ionic drug by being impregnated with the ionic drug, the drug solution holding portion being placed adjacent to the first ion exchange membrane, a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion, and a shape-memory separator capable of being transformed between a first and a second configuration to respectively allow passage of a substance and blocking of the substance, the shape-memory separator placed adjacent to at least one surface of the first ion exchange membrane.

### BACKGROUND

1. Technical Field

The present invention disclosure relates to a technique (transdermal drug delivery) for transdermally administering various ionic drugs by means of iontophoresis. More specifically, the present invention disclosure relates to an electrode assembly to be used for iontophoresis and an iontophoresis device using the same.

2. Description of the Related Art

A method of introducing (permeating) an ionic drug placed on the surface of the skin or mucosa (hereinafter, merely referred to as “skin”) of a predetermined site of an organism into the body through the skin by giving the skin an electromotive force sufficient to drive such an ionic drug is called iontophoresis (iontophorese, ion introduction method, ion permeation therapy) (See e.g., JP 63-35266 A).

For example, positively charged ions are driven (transported) into the skin on the side of an anode (positive electrode) in an electric system of an iontophoresis device. On the other hand, negatively charged ions are driven (transported) into the skin on the side of a cathode (negative electrode) in the electric system of the iontophoresis device.

A large number of such iontophoresis devices as described above have been conventionally proposed (See e.g., JP 63-35266 A, JP 04-297277 A, JP 2000-229128 A, JP 2000-229129 A, JP 2000-237327 A, JP 2000-237328 A and WO 03/037425 A1). Some of those documents propose, as an electrode assembly for iontophoresis, an electrode assembly obtained by laminating: an electrode; an electrolyte solution holding portion; an ion exchange membrane selecting an ion having polarity opposite to that of an ionic drug; a drug solution holding portion holding the ionic drug by being impregnated with the ionic drug; and an ion exchange membrane selecting an ion having the same polarity as that of the ionic drug.

However, during the period commencing on the production of the electrode assembly and ending on the use of the assembly, a component in the electrolyte solution or a component in the drug solution (mainly an ion component having polarity opposite to that of the ionic drug) may move through the ion exchange membrane selecting an ion having polarity opposite to that of the ionic drug. A certain component in the electrolyte solution or a certain component in the drug solution may cause an adverse effect (such as the alteration of a drug component, a reduction in stability of the drug, a reduction in amount of the drug that can be released, or a reduction in transport number due to the mixing of a dissimilar ion).

Therefore, preventing the movement of a substance between the electrolyte solution and the drug solution until the electrode assembly for iontophoresis is used, and enabling the movement of a certain substance at the time of use are important problems.

### BRIEF SUMMARY

Some embodiment of the present invention provide an electrode assembly for iontophoresis including a separator to prevent the movement of a substance between an electrolyte solution and a drug solution until the electrode assembly for iontophoresis is used and enabling the movement of a certain substance at the time of use, and an iontophoresis device using the same.

According to one embodiment, an electrode assembly for iontophoresis includes an electrode coupled to an electric power source device having a same polarity as that of the ionic drug in the electrode assembly, an electrolyte solution holding portion holding an electrolyte solution by being impregnated with the electrolyte solution, the electrolyte solution holding portion being placed adjacent to the electrode, a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion, a drug solution holding portion holding the ionic drug by being impregnated with the ionic drug, the drug solution holding portion being placed adjacent to the first ion exchange membrane, a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion, and a shape-memory separator capable of being transformed between a first and a second configuration to respectively allow passage of a substance and blocking of the substance, the shape-memory separator placed adjacent to at least one surface of the first ion exchange membrane.

According to another embodiment, an iontophoresis device includes an electric power source device, a drug administration device including two or more electrode assemblies which include one or more electrode assemblies having: an electrode coupled to the electric power source device having a same polarity as that of an ionic drug in the electrode assembly, an electrolyte solution holding portion holding an electrolyte solution by being impregnated with the electrolyte solution, the electrolyte solution holding portion being placed adjacent to the electrode, a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion, a drug solution holding portion holding the ionic drug by being impregnated with the ionic drug, the drug solution holding portion being placed adjacent to the first ion exchange membrane, a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion, and a shape-memory separator capable of being transformed between a first and a second deformation to respectively allow passage of a substance and blocking of the substance, the shape-memory separator placed adjacent to at least one surface of the first ion exchange membrane, the drug administration device coupled to the electric power source device, and a current control device to control a current flowing to each of the electrode assemblies, wherein the ionic drug is released from each of the electrode assemblies and transdermally administered to an organism in accordance with the current flowing from the current control device.

According to yet another embodiment, an electrode assembly for iontophoresis includes an electrode coupled to an electric power source device having a same polarity as that of the ionic drug in the electrode assembly, an electrolyte solution holding portion holding an electrolyte solution by being impregnated with the electrolyte solution, the electrolyte solution holding portion being placed adjacent to the electrode, a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion, a drug solution holding portion holding the ionic drug by being impregnated with the ionic drug, the drug solution holding portion being placed adjacent to the first ion exchange membrane, a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion, and a shape-memory separator capable of being transformed between a first and a second configuration to respectively allow passage of a substance and blocking of the substance, the shape-memory separator placed adjacent to at least one surface of the second ion exchange membrane.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 shows a cross-sectional schematic illustration of an electrode assembly for iontophoresis, according to one illustrated embodiment of the present invention.

FIG. 2 shows a cross-sectional schematic illustration of an iontophoresis device including the electrode assembly for iontophoresis, according to one illustrated embodiment of the present invention.

### DETAILED DESCRIPTION

As described above, in the electrode assembly for iontophoresis according to one embodiment of the present invention, the shape-memory separator capable of switching the transmission and blocking of a substance through the deformation of the shape-memory resin is arranged adjacent to at least one surface of the ion exchange membrane selecting an ion having polarity opposite to that of the ionic drug. As a result, the movement of a substance between the electrolyte solution and the drug solution can be prevented until the electrode assembly for iontophoresis is used, and the movement of a certain substance is enabled at the time of use.

Hereinafter, embodiments of the present invention will be described on the basis of specific examples shown in the drawings.

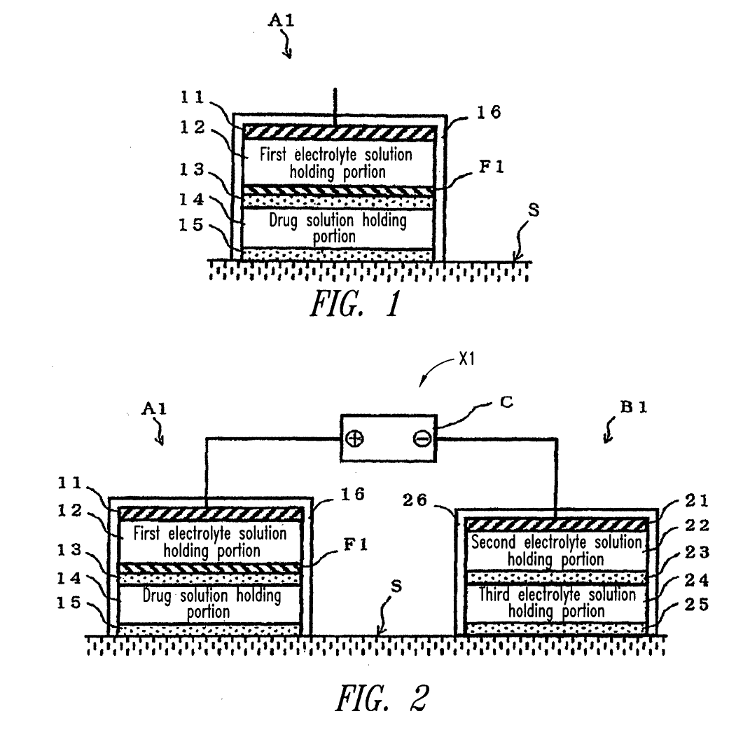

FIG. 1 is a schematic view showing a state where an electrode assembly A1 for iontophoresis according to one embodiment of the present invention which is arranged on a skin S is used. The electrode assembly A1 is used as a working electrode assembly for transdermally administering an ionic drug in an iontophoresis device. The electrode assembly A1 for iontophoresis may includes a first electrode 11 coupled to an electric power source device having the same polarity as that of the charge of an ionic drug through an electric cable. A first electrolyte solution holding portion 12 may hold an electrolyte solution by being impregnated with the electrolyte solution and may be arranged adjacent to the first electrode 11 A shape-memory separator F1 may be arranged adjacent to the first electrolyte solution holding portion 12. A first ion exchange membrane 13 may substantially pass ions having a polarity that is same as a polarity of the ionic drug and substantially block ions having a polarity that is opposite the polarity of the ionic drug. The first ion exchange membrane 13 may be arranged adjacent to the separator F1. A drug solution holding portion 14 may hold the ionic drug by being impregnated with the ionic drug. The drug solution holding portion 14 may be arranged adjacent to the first ion exchange membrane 13. A second ion exchange membrane 15 may substantially pass ions having a polarity opposite the polarity of the ionic drug and substantially block ions having a polarity that is the same as a polarity of the ionic drug. The second ion exchange membrane 15 may be arranged adjacent to the drug solution holding portion 14. The first electrode 11, the first electrolyte solution holding portion 12, the first ion exchange membrane 13, the drug solution holding portion 14 and the second ion exchange membrane 15 may be housed in a cover 16.

FIG. 2 is a cross-sectional schematic view showing an iontophoresis device X1 including the electrode assembly (i.e., working electrode assembly) A1 useful for iontophoresis according to one embodiment of the present invention, an electric power source device C and a non-working electrode assembly B1 serving as a counter electrode assembly to the electrode assembly A1. The iontophoresis device X1 may be arranged on the skin S.

The electrode assembly A1 useful for iontophoresis may be coupled to the same polarity of the electric power source device C as that of the ionic drug via an electric wire. In addition, the non-working electrode assembly B1 may include a second electrode 21 coupled to the polarity of the electric power source device C opposite that of the ionic drug via an electric wire. A second electrolyte solution holding portion 22 may hold an electrolyte solution by being impregnated with the electrolyte solution and may be arranged adjacent to the second electrode 21. A third ion exchange membrane 23 may substantially pass ions having a polarity opposite the polarity of the ionic drug and substantially blocks ions having a polarity that is the same as the polarity of the ionic drug. The third ion exchange membrane 23 may be arranged adjacent to the second electrolyte solution holding portion 22. A third electrolyte solution holding portion 24 may hold an electrolyte solution by being impregnated with the electrolyte solution and may be arranged adjacent to the third ion exchange membrane 23. A fourth ion exchange membrane 25 substantially passes ions having a polarity that is same as the polarity of the ionic drug and substantially blocks ions having a polarity opposite the polarity of the ionic drug. The fourth ion exchange membrane 25 may be arranged adjacent to the third electrolyte solution holding portion 24. The second electrode 21, the second electrolyte solution holding portion 22, the third ion exchange membrane 23, the third electrolyte solution holding portion 24 and the fourth ion exchange membrane 25 may be housed in the cover 16. The above non-working electrode assembly B1 is exemplified as one embodiment, and is not limited to the above embodiment.

In FIG. 2, the electrode assembly A1 for iontophoresis is coupled to a positive side of the electric power source device C while the non-working electrode assembly B1 is coupled to a negative side of the electric power source device C. Of course, the electrode assembly A1 and the non-working electrode assembly B1 may be connected to the negative side and the positive side, respectively, depending on the polarity of the ionic drug.

In the iontophoresis device X1, when the electrode assembly A1 holding the ionic drug is energized by the electric power source C, the ionic drug moves to a side opposite the first electrode 11 as a result of electrophoresis by virtue of an electric field, and is transdermally administered to an organism via the second ion exchange membrane 15. At such time, the first ion exchange membrane 13 arranged on the electrode side substantially passes ions having a polarity that is the same as the ionic drug and substantially blocks ions having a polarity that is opposite the polarity of the ionic drug, thereby preventing the movement of the ionic drug to the first electrode 11 side. Meanwhile, the second ion exchange membrane 15 arranged on the skin S substantially passes ions having a polarity opposite the polarity of the ionic drug and substantially blocks ions having a polarity that is the same as a polarity of the ionic drug. As a result, the ionic drug can be efficiently released, whereby the ionic drug can be administered to the skin S at a high transport number. Furthermore, the composition of the electrode assembly A1, as described above, prevents damage to the skin S based on an electrochemical reaction and the ionic drug can be safely administered. In addition, the following conditions may, for example, be adopted as energizing conditions in the iontophoresis device X1: a constant current condition of for example, 0.1 mA/cm² to 0.5 mA/cm² or 0.1 0.3 mA/cm² to 0.3 mA/cm², and a safe voltage condition that realizes the above constant current. The safe voltage condition may, for example, be 50 V or less or 30 V or less.

According to one embodiment of the present invention, multiple working electrode assemblies or multiple non-working electrode assemblies may be included in the iontophoresis device X1. In such case, one working electrode assembly may be caused to hold multiple kinds of ionic drugs. When multiple ionic drugs different from each other in polarity are to be administered, a working electrode assembly and a non-working electrode assembly may be arranged on an anode side, and a working electrode assembly and a non-working electrode assembly may be arranged also on a cathode side.

Alternatively, multiple electrode assemblies may serve as a drug administering means and assembled in one package to achieve, for example, convenience of handling. A material to be used for the package in this case is not particularly limited as long as it does not affect the administration of an ionic drug, and an example of such material includes polyolefin for medical equipment. Furthermore, a current control means may be arranged for administering a defined amount of a drug within a defined time period. The drug administering means, the current control means, and an electric power source device may be integrally formed by providing the electric power source device as, for example, a button battery and the current control means as, for example, an integrated circuit for miniaturization.

The separator F1 to be used for the electrode assembly may be a shape-memory separator capable of switching the transmission and blocking of a substance through the deformation of a shape-memory resin, and may be arranged adjacent to at least one surface of the ion exchange membrane. The ion exchange membrane may substantially pass ions having a polarity that is same as a polarity of the ionic drug and substantially block ions having a polarity that is opposite the polarity of the ionic drug. In FIG. 1, the separator F1 may be arranged adjacent to the side of the first electrolyte solution holding portion 12 of the first ion exchange membrane 13. Alternatively, the separator F1 may be arranged on the side of the drug solution holding portion 14, or two separators F1 may be respectively arranged adjacent the side of the first electrolyte solution holding portion 12 and the drug solution holding portion 14.

In another embodiment of the present invention, the shape-memory separator F1 can be arranged adjacent to at least one surface of the ion exchange membrane which may substantially pass ions having a polarity opposite the polarity of the ionic drug and substantially block ions having a polarity that is the same as a polarity of the ionic drug. As such, the administration itself of a drug can be controlled with the separator F1, the passage of the drug can be permitted only when the passage is needed, and the efficient administration of the drug can be realized with improved sureness.

The term “shape-memory resin (shape-memory polymer)” as used herein typically refers to, for example, a resin which can be deformed and processed in a defined temperature range (e.g., temperatures equal to or higher than a glass transition temperature), which is immobilized at low temperatures, and which can return to its original shape when heated again to the defined temperature range (e.g., temperatures equal to or higher than the glass transition temperature). Some degree of shape-memory property may be inherent in any one of most polymer materials.

The separator F1 may be formed of the membrane of a shape-memory resin that can serve as a porous body or a porous membrane containing a shape-memory resin. Upon production or storage of the electrode assembly, the pores of the separator F1 are closed so that the movement of a substance is blocked. The separator F1 deforms to a porous body in response to a certain stimulus to permit the transmission of the substance. The substance whose transmission is permitted may include at least an ion operable to pass through an ion exchange membrane, and any other substance may pass. Providing such separator F1 can prevent the movement of a substance between the electrolyte solution and the drug solution until the electrode assembly for iontophoresis is used, and can enable the movement of the substance at the time of use. Accordingly, it becomes possible to prevent or otherwise reduce an adverse effect due to the movement of a component in the electrolyte solution or a component in the drug solution (e.g., an ion component having polarity opposite that of the ionic drug) through the ion exchange membrane during the period commencing on the production of the assembly and ending on the use of the assembly.

Examples of the above-described certain stimulus for deforming the shape-memory resin include heat (temperature) and an electric stimulus. For example, in the case where a shape-memory resin which blocks the transmission of a substance at a temperature lower than 30° C. and which deforms to be porous when heated to 30° C. or higher to thereby permit the transmission of the substance is used, a separator can be obtained, which blocks the transmission of a substance while the electrode assembly is stored in a cold space, and which, when the electrode assembly is mounted on an organism, causes the substance to transmit by being heated with the body temperature. In addition, for example, in the embodiment where a shape-memory resin which blocks the transmission of a substance when no voltage is applied and which deforms to be porous in response to the application of a voltage to permit the transmission of the substance is used, a separator can be obtained, which blocks the transmission of a substance while the electrode assembly is stored, and which causes the substance to transmit when a voltage is applied to start the administration of a drug by means of iontophoresis.

The deformation of the shape-memory resin due to the certain stimulus may be reversible or irreversible. When a shape-memory resin that irreversibly deforms is used, the transfer of a substance through the separator F1 may be permitted by applying an initial stimulus of a substantial strength to the shape-memory resin prior to mounting the electrode assembly A1 on an organism as long as a constituent of the electrode assembly A1 such as a drug can withstand the stimulus. For example, the transfer of a substance through the separator F1 may be permitted by initially heating the separator up to approximately 40° C. immediately before use, or the transfer of a substance through the separator F1 may be permitted by applying an initial voltage of 100 V to the separator immediately before use. After that, mounting the electrode assembly A1 on the organism allows the administration of the drug by means of iontophoresis to start. In embodiments where a shape-memory resin that reversibly deforms is used, the transfer of a substance through the separator F1 may be permitted upon administration in the case where a drug is intermittently administered.

Any shape-memory resin can be used for the separator F1 in one embodiment of the present invention without any particular limitation as long as the resin restores its shape under a defined condition. Examples of such resin may include polyester, polyurethane, styrene.butadiene, polynorbornene, transpolyisoprene, poly N-isopropylacryalmide, and an ethylene glycol-propylene glycol copolymer.

The separator F1 may, for example, be of 1 μm to 1 mm in thickness and 0.01 μm to 100 μm in pore size. Upon formation of a shape-memory separator, for example, after a granular shape-memory resin has been subjected to compression or the like to provide a porous material, or after a shape-memory resin has been foamed to provide a porous material, the resultant porous material is compressed in a defined temperature range so that continuous air bubbles disappear. As a result, the resultant blocks the transmission of a substance at a temperature equal to or lower than a defined temperature because the resultant has no continuous air bubbles, but returns to a porous material having continuous air bubbles when heated to a temperature equal to or higher than the defined temperature, to thereby permit the transmission of the substance.

A specific example of such shape-memory separator F1 may include such porous film as described in AlChE Journal Vol. 49, No. 4, p. 896 to 909, that is, a polyethylene porous film having a thickness of 100 μm, a pore size of 0.28 μm, and a porosity of 69% to which graft poly(N-isopropylacrylamide) (PNIPAM) as a temperature-responsive polymer may be caused to adhere by means of a plasma•graft pore filling polymerization method so that transmittance is controlled in terms of temperature.

An inactive electrode made of a conductive material such as carbon or platinum can be used as the electrode 11 of the electrode assembly A1.

The first electrolyte solution holding portion 12 used for the electrode assembly A1 may include a thin film that has the property of holding an electrolyte solution by being impregnated with the electrolyte solution. The thin film can be made of the same material as that used for the drug solution holding portion 15 for holding an ionic drug by being impregnated with the ionic drug to be described later. A desired one can be appropriately used as the electrolyte solution depending upon the conditions such as a drug to be applied. However, it may be desirable to avoid an electrolyte solution that damages the skin of an organism in response to an electrode reaction. An organic acid or a salt thereof present in a metabolic cycle of an organism may be used as the electrolyte solution in one embodiment of the present invention in consideration of harmlessness. For example, lactic acid and fumaric acid may be used. Specifically, an aqueous solution of 1M of lactic acid and 1M of sodium fumarate (1:1) may be used. Such electrolyte solution may be used because: it has high solubility with respect to water and passes a current well; and in the case where a current is allowed to flow at a constant level, the electric resistance is low and a change in pH is relatively small in an electric power source device.

In general, an electrolyte solution that does not interact with a drug may be used. However, in embodiments of the present invention even an electrolyte solution that causes an interaction with a drug such as the alteration of a drug component, a reduction in stability of the drug, a reduction in amount of the drug that can be released, or a reduction in transport number due to the mixing of a dissimilar ion can be suitably used.

The drug solution holding portion 14 may include a thin film that holds an ionic drug or the like by being impregnated with the ionic drug or the like. The thin film having substantial ability to hold the ionic drug or the like by being impregnated with the ionic drug or the like, and a substantial ability to transfer (i.e., ion transferability, ion conductivity) an ionic drug impregnated into and held by the thin film to the skin S side while under defined electric field conditions. Examples of a material that brings together good property of holding a drug by being impregnated with the drug and good ion conductivity may include hydrogel forms of acrylic resins (e.g., acrylic hydrogel film), a segmented polyurethane-based gel film, and an ion-conductive porous sheet to form a gel-like solid electrolyte (e.g., a porous polymer disclosed in JP 11-273452 A using, as a base, an acrylonitrile copolymer having 50 mol % or more, or 70 mol % to 98 mol % or more of acrylonitrile and having a porosity of 20% to 80%). When the drug solution holding portion 14 as described above is impregnated with a drug, an impregnation rate (defined by 100×(W−D)/D (%) where D indicates a dry weight and W indicates a weight after impregnation) may approximately be 30% to 40%.

A cation exchange membrane and an anion exchange membrane are preferably used together as ion exchange membranes 13, 15 to be used for the electrode assembly A1. Examples of the cation exchange membrane may include NEOSEPTAs (CM-1, CM-2, CMX, CMS, CMB, and CLE04-2) manufactured by Tokuyama Co., Ltd. Examples of the anion exchange membrane may include NEOSEPTAs (AM-1, AM-3, AMX, AHA, ACH, ACS, ALE04-2, and AIP-21) manufactured by Tokuyama Co., Ltd. Other examples may include: an ion exchange membrane that includes a porous film having cavities a part or whole of which are filled with an ion exchange resin having a cation exchange function; and an ion exchange membrane that includes a porous film having cavities a part or whole of which are filled with an ion exchange resin having an anion exchange function.

The above-mentioned ion exchange resins may be fluorine-based that include a perfluorocarbon skeleton having an ion exchange group and may be hydrocarbon-based that include a nonfluorinated resin as a skeleton. From the viewpoint of convenience of production process, hydrocarbon-based ion exchange resins may be used. The filling rate of the porous film with the ion exchange resin, which varies depending on the porosity of the porous film, may be, for example, 5 to 95 mass %, 10 to 90 mass %, or 20 to 60 mass %.

In addition, the ion exchange group in the above-mentioned ion exchange resin is not particularly limited in so far as it is a functional group that generates a group having negative or positive charge in aqueous solutions. Such functional group may be present in the form of a free acid or a salt. Examples of a cation exchange group may include a sulfonic group, a carboxylic acid group, and a phosphonic acid group. Of those, a sulfonic group may be used. Examples of a counter cation for the cation exchange group may include alkali cations such as, for example, a sodium ion and a potassium ion; and ammonium ions. Examples of an anion exchange group may include a primary amino group, a secondary amino group, a tertiary amino group, a quaternary amino group, a pyridyl group, an imidazole group, a quaternary pyridium group, and a quaternary imidazolium group. Of those, a quaternary ammonium group or a quaternary pyridium group may be used. Examples of a counter cation for the anion exchange group may include halogen ions such as a chlorine ion and hydroxy ions.

In addition, the above-mentioned porous film is not particularly limited and any porous film can be used in so far as it is in the form of a film or sheet that has a large number of pores communicating with both sides thereof. To satisfy both of high strength and flexibility, the porous film may be made of a thermoplastic resin. Examples of the thermoplastic resin comprising the porous film may include: polyolefin resins such as, for example, homopolymers or copolymers of α-olefins such as ethylene, propylene, 1-butene, 1-pentene, 1-hexene, 3-methyl-1-butene, 4-methyl-1-pentene, and 5-methyl-1-heptene; vinyl chloride-based resins such as polyvinyl chloride, vinyl chloride-vinyl acetate copolymers, vinyl chloride-vinylidene chloride copolymers, and vinyl chloride-olefin copolymers; fluorine-based resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene-ethylene copolymers; polyamide resins such as nylon 66; and polyimide resins. Of those, polyolefin resins may be used when considering, for example, mechanical strength, flexibility, chemical stability, and chemical resistance. Of those, polyethylene or polypropylene may be used. In some embodiments, polyethylene may be used.

Further, the mean pore size of the above-mentioned porous film made of the thermoplastic resin may, for example, be 0.005 μm to 5.0 μm, 0.01 μm to 2.0 μm, or 0.02 μm to 0.2 μm. It is noted that the above-mentioned exemplary mean pore sizes as used herein means a mean flow pore size measured in conformance with the bubble point method (e.g., JIS K3832-1990).

In addition, the porosity of the porous film may, for example, be 20% to 95%, 30% to 90%, or 30% to 60%. In consideration of the thickness of an ion exchange membrane to be finally formed, the thickness of the porous film may, for example, be 5 μm to 140 μm, 10 μm to 130 μm, or 15 μm to 55 μm. An anion exchange membrane or a cation exchange membrane formed of such porous film may have the same thickness as that of the porous film or up to about 20 μm larger than the thickness of the porous film.

As described above, the electrode assembly A1 for iontophoresis, according to embodiments of the present invention, may hold an ionic drug.

Examples of the ionic drug may include: anesthetic drugs (e.g., procaine hydrochloride and lidocaine hydrochloride), gastrointestinal disease therapeutic (e.g., carnitine chloride), skeletal muscle relaxants (e.g., vancuronium bromide) and antibiotics (e.g., a tetracycline-based preparation, a kanamycin-based preparation, and a gentamicin-based preparation).

Examples of the ionic drug that can be negatively charged may include: vitamin (e.g., riboflavin sodium, nicotine acid, ascorbic acid, and folic acid), adrenal cortex hormones (e.g., a hydrocortisone-based water-soluble preparation, a dexamethasone-based water-soluble preparation, and a prednisolone-based water-soluble preparation such as prednisolone sodium phosphate and dexamethasone sodium phosphate) and antimicrobial drug (e.g., a quinolone-based preparation).

Examples of a vaccine may include a BCG vaccine, a hepatitis A vaccine, a melanoma vaccine, a measles vaccine, a poliomyelitis vaccine, and an influenza vaccine.

Examples of an adjuvant may include MPL (Monophosphoryl lipid A), DMPC (dimyristoylphosphatidylcholine), QS-21, DDA (Dimethyl dioctadecyl ammonium chloride), and RC-529.

Furthermore, examples of a combination of a vaccine and an adjuvant may include: a combination of a positively ionized vaccine and RC-529; a combination of a negatively ionized vaccine and DDA; a combination of a BCG vaccine and MPL; a combination of a hepatitis A vaccine and DMPC; and a combination of a melanoma vaccine and QS-21.

In addition to the above combinations of vaccines and adjuvants, examples of a combination of drugs may include: a combination of a hypotensive drug and a hypotensive diuretic agent such as a combination of lisinopril and hydrochlorothiazide, a combination of methyldopa and hydrochlorothiazide, a combination of clonidine hydrochloride and chlorthalidone, or a combination of benazepril hydrochloride and hydrochlorothiazide; a combination of antidiabetic agents such as a combination of insulin and metformin hydrochloride; and any other combination such as a combination of ozagrel hydrochloride and ozagrel sodium or a combination of codeine hydrochloride and promethazine hydrochloride.

In addition, multiple kinds of ionic drugs to be held by the electrode assembly A1 for iontophoresis according to some embodiments of the present invention may be appropriately combined depending on, for example, the kind of a disease and the condition of a patient. This means that different ionic drugs may be held by electrode assemblies or multiple kinds of ionic drugs may be combined in a single electrode assembly.

The amount of an ionic drug is determined for each individual ionic drug in such a manner that an effective blood concentration preset upon application to a patient can be obtained for an effective time period. The amount is set by one skilled in the art in accordance with, for example, the size and thickness of a drug solution holding portion or the like, the area of a drug release surface, a voltage in an electrode device, and an administration time.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An electrode assembly for iontophoresis holding an ionic drug, the electrode assembly comprising:
an electrode coupled to an electric power source device having a same polarity as that of the ionic drug in the electrode assembly;
an electrolyte solution holding portion holding an electrolyte solution by being impregnated with the electrolyte solution, the electrolyte solution holding portion being placed adjacent to the electrode;
a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion;
a drug solution holding portion holding the ionic drug by being impregnated with the ionic drug, the drug solution holding portion being placed adjacent to the first ion exchange membrane;
a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion; and
a shape-memory separator capable of being transformed between a first and a second configuration to respectively allow passage of a substance and blocking of the substance, the shape-memory separator placed adjacent to at least one surface of the first ion exchange membrane.

2. The electrode assembly for iontophoresis according to claim 1, wherein
the shape-memory separator takes a form of at least one of a membrane of a shape-memory resin capable of being porous and a porous membrane including the shape-memory resin,
the shape-memory separator closes a pore to block movement of the substance prior to use, and transforms to being porous in response to at least one of being heated to a defined temperature range and being applied with a desired voltage to allow the substance to pass therethrough.

3. The electrode assembly for iontophoresis according to claim 2, wherein the shape-memory separator blocks the passage of the substance in response to being at a temperature lower than 30° C. and transforms into a porous state to allow the substance to pass therethrough in response to being heated to a temperature of 30° C. or higher.

4. The electrode assembly for iontophoresis according to claim 2, wherein the shape-memory separator becomes porous to allow the substance to pass in response to deformation of the shape-memory resin caused by heating the shape-memory separator to a temperature of approximately 40° C. or higher, the shape-memory separator operable to maintains passage of the substance while being cooled to a temperature of lower than 40° C.

5. An iontophoresis device comprising:
an electric power source device;
a drug administration device including two or more electrode assemblies which include one or more electrode assemblies having:
an electrode coupled to the electric power source device having a same polarity as that of an ionic drug in the electrode assembly,
an electrolyte solution holding portion holding an electrolyte solution by being impregnated with the electrolyte solution, the electrolyte solution holding portion being placed adjacent to the electrode,
a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion,
a drug solution holding portion holding the ionic drug by being impregnated with the ionic drug, the drug solution holding portion being placed adjacent to the first ion exchange membrane,
a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion, and
a shape-memory separator capable of being transformed between a first and a second deformation to respectively allow passage of a substance and blocking of the substance, the shape-memory separator placed adjacent to at least one surface of the first ion exchange membrane, the drug administration device coupled to the electric power source device; and
a current control device to control a current flowing to each of the electrode assemblies,
wherein the ionic drug is released from each of the electrode assemblies and transdermally administered to an organism in accordance with the current flowing from the current control device.

6. An electrode assembly for iontophoresis holding an ionic drug, the electrode assembly comprising:
an electrode coupled to an electric power source device having a same polarity as that of the ionic drug in the electrode assembly;
an electrolyte solution holding portion holding an electrolyte solution by being impregnated with the electrolyte solution, the electrolyte solution holding portion being placed adjacent to the electrode;
a first ion exchange membrane operable to substantially pass ions having a polarity that is same as a polarity of the ionic drug and substantially block ions having a polarity that is opposite the polarity of the ionic drug, the first ion exchange membrane being placed adjacent to the electrolyte solution holding portion;
a drug solution holding portion holding the ionic drug by being impregnated with the ionic drug, the drug solution holding portion being placed adjacent to the first ion exchange membrane;
a second ion exchange membrane operable to substantially pass ions having a polarity that is opposite the polarity of the ionic drug and substantially block ions having a polarity that is the same as the polarity of the ionic drug, the second ion exchange membrane being placed adjacent to the drug solution holding portion; and
a shape-memory separator capable of being transformed between a first and a second configuration to respectively allow passage of a substance and blocking of the substance, the shape-memory separator placed adjacent to at least one surface of the second ion exchange membrane.
